(19) United States
(12) Patent Application Publication
(10) Pub. No.: US 2024/0239731 A1
(43) Pub. Date: Jul. 18, 2024

(54) Mixture of two constitutionally isomeric bisphosphites

(71) Applicant: Evonik Oxeno GmbH & Co. KG, Marl (DE)

(72) Inventors: Anna Chiara Sale, Recklinghausen (DE); Robert Franke, Marl (DE); Alexander Brächer, Hackenheim (DE); Dirk Fridag, Haltern am See (DE); Johannes Knossalla, Everswinkel (DE); Peter Kucmierczyk, Herne (DE); Ana Markovic, Haltern am See (DE)

(73) Assignee: Evonik Oxeno GmbH & Co. KG, Marl (DE)

(21) Appl. No.: 18/410,227

(22) Filed: Jan. 11, 2024

(30) Foreign Application Priority Data
Jan. 18, 2023 (EP) 23152173.3

(51) Int. Cl. C07C 45/50; B01J 31/18; B01J 31/20; B01J 31/22

(52) U.S. Cl. CPC C07C 45/505; B01J 31/185; B01J 31/20; B01J 31/2208; B01J 2231/321; B01J 2531/004; B01J 2531/822

(57) ABSTRACT

Mixture of two constitutionally isomeric bisphosphites and use thereof in hydroformylation.

The Invention relates to a mixture of two constitutionally isomeric bisphosphites and use thereof in hydroformylation.

EP 2 906 572 B1 describes a mixture of constitutionally isomeric bisphosphites (2a+2b).

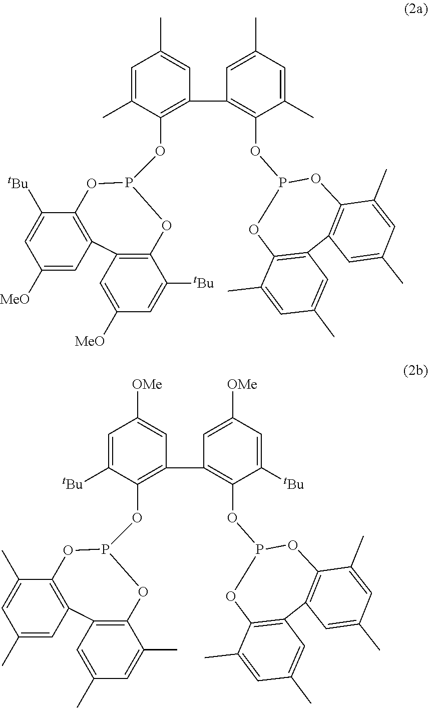 [SMILES: COc1cc(C(C)(C)C)c2op(Oc3c(C)cc(C)cc3-c3cc(C)cc(C)c3Op3oc4c(C)cc(C)cc4c4cc(C)cc(C)c4o3)oc3c(C(C)(C)C)cc(OC)cc3c2c1 ; COc1cc(-c2cc(OC)cc(C(C)(C)C)c2Op2oc3c(C)cc(C)cc3c3cc(C)cc(C)c3o2)c(Op2oc3c(C)cc(C)cc3c3cc(C)cc(C)c3o2)c(C(C)(C)C)c1]

The technical object of the invention is to provide a mixture with which an increased yield can be achieved in the hydroformylation of olefins.

The object is achieved by a mixture according to claim 1.

Mixture comprising the compounds (1a) and (1b):

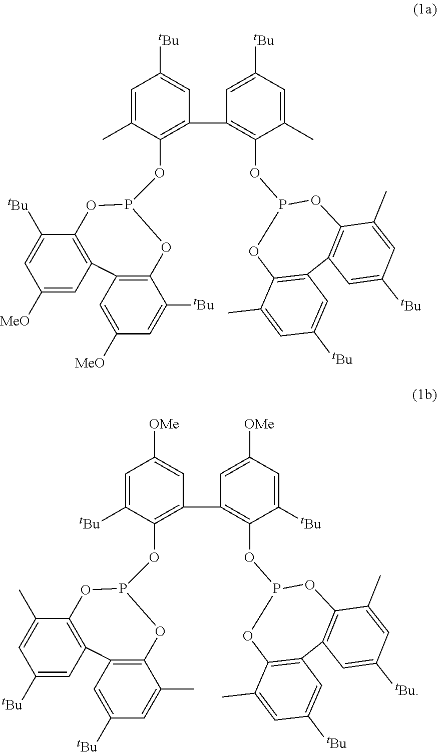 [SMILES: COc1cc(C(C)(C)C)c2op(Oc3c(C)cc(C(C)(C)C)cc3-c3cc(C(C)(C)C)cc(C)c3Op3oc4c(C)cc(C(C)(C)C)cc4c4cc(C(C)(C)C)cc(C)c4o3)oc3c(C(C)(C)C)cc(OC)cc3c2c1 ; COc1cc(-c2cc(OC)cc(C(C)(C)C)c2Op2oc3c(C)cc(C(C)(C)C)cc3c3cc(C(C)(C)C)cc(C)c3o2)c(Op2oc3c(C)cc(C(C)(C)C)cc3c3cc(C(C)(C)C)cc(C)c3o2)c(C(C)(C)C)c1]

In addition to the mixture itself, a process in which the mixture is used is also claimed.

Process comprising the process steps of:
a) initially charging an olefin;
b) adding a mixture described above;
c) adding a Rh compound;
d) feeding in H₂ and CO,
e) heating the reaction mixture from a) to d), to convert the olefin to an aldehyde.

In one variant of the process, the olefin in process step a) is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

In one variant of the process, the Rh compound is selected from: Rh(acac)(CO)₂, [(acac)Rh(COD)] (Umicore, acac=acetylacetonate anion; COD=1,5-cyclooctadiene), Rh₄CO₁₂.

In one variant of the process, the Rh compound is Rh(acac)(CO)₂.

In one variant of the process, the H₂ and CO in process step d) are fed in at a pressure in the range from 1 to 6 MPa (10 to 60 bar).

In one variant of the process, the heating of the reaction mixture in process step e) is to a temperature in the range from 80° C. to 160° C.

The invention is to be illustrated in more detail hereinafter by a working example.

### Catalysis Experiments

The hydroformylation was carried out in a 16 ml autoclave from HEL Group, Hertfordshire, Great Britain, equipped with a constant-pressure apparatus, gas flowmeter and sparging stirrer. The substrate (1-octene) was heated under reflux over sodium for several hours and distilled under argon.

The reaction solutions for the experiments were prepared beforehand under an argon atmosphere. For this purpose, 100 ppm Rh(acac)(CO)₂ and the corresponding amount of phosphite compound (L:Rh=50:1) were weighed out and made up with 8.0 ml of toluene. The mass of toluene introduced in each case was determined for the GC analysis. 1.80 g of 1-octene (16 mmol) were then added. The prepared solutions were then introduced into the autoclave and said autoclave was purged three times with argon and three times with synthesis gas (Linde; H₂ (99.999%):CO (99.997%)=1:1). The autoclave was then heated to the desired temperature at an overall pressure of 10 bar with stirring (900 rpm). Upon achieving the reaction temperature, the synthesis gas pressure was increased to 17 bar and the reaction was carried out for 1 h at constant pressure. Once the reaction time had elapsed the autoclave was cooled to room temperature, decompressed with stirring and purged with argon. 0.5 ml of each reaction mixture was withdrawn after termination of the reaction, diluted with 4 ml of pentane and analysed by gas chromatography: HP 5890 Series II plus, PONA, 50 m×0.2 mm×0.5 μm. Residual olefin and aldehyde were quantitatively determined against the solvent toluene as internal standard.

The experiment was carried out with the mixtures (1a+1b) and (2a+2b).

The mixture (2a+2b) serves as a comparison here.

### Results of the Catalysis Experiments

[Rh]: 100 ppm, p: 17 bar, T: 120° C.; t: 1 h

TABLE
Hydroformylation of 1-octene

| Ligand | Yield [%] |
|---|---|
| (1a + 1b)* | 24 |
| (2a + 2b) | 16 |

*inventive mixture

The experiments carried out demonstrate that the object assigned is achieved by a mixture according to the invention.

1. Mixture comprising the compounds (1a) and (1b):

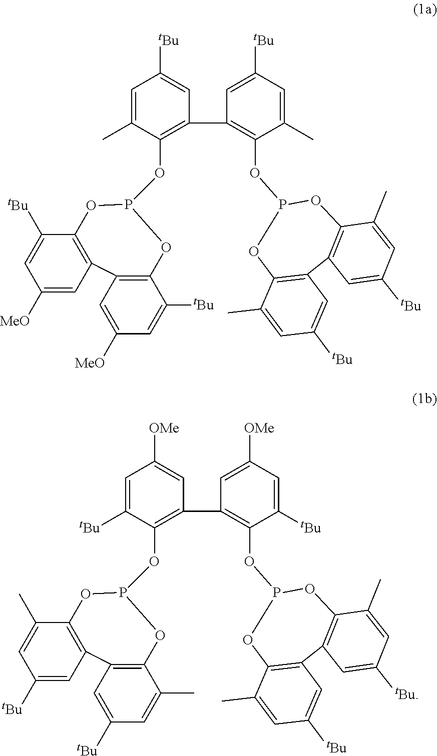 [SMILES: COc1cc(C(C)(C)C)c2op(Oc3c(C)cc(C(C)(C)C)cc3-c3cc(C(C)(C)C)cc(C)c3Op3oc4c(C)cc(C(C)(C)C)cc4c4cc(C(C)(C)C)cc(C)c4o3)oc3c(C(C)(C)C)cc(OC)cc3c2c1 ; COc1cc(-c2cc(OC)cc(C(C)(C)C)c2Op2oc3c(C)cc(C(C)(C)C)cc3c3cc(C(C)(C)C)cc(C)c3o2)c(Op2oc3c(C)cc(C(C)(C)C)cc3c3cc(C(C)(C)C)cc(C)c3o2)c(C(C)(C)C)c1]

2. Process comprising the process steps of:
a) initially charging an olefin;
b) adding a mixture according to claim 1;
c) adding a Rh compound;
d) feeding in H₂ and CO,
e) heating the reaction mixture from a) to d), to convert the olefin to an aldehyde.

3. Process according to claim 2,
wherein the olefin in process step a) is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

4. Process according to claim 2,
wherein the Rh compound is selected from: Rh(acac)(CO)₂, [(acac)Rh(COD)] (Umicore, acac=acetylacetonate anion; COD=1,5-cyclooctadiene), Rh₄CO₁₂.

5. Process according to claim 2,
wherein the Rh compound is Rh(acac)(CO)₂.

6. Process according to claim 2,
wherein the H₂ and CO in process step d) is fed in at a pressure in the range of 1 to 6 MPa (10 to 60 bar).

7. Process according to claim 2,
wherein the heating of the reaction mixture in process step e) is to a temperature in the range of 80° C. to 160° C.
